(19) United States
(12) Patent Application Publication
(10) Pub. No.: US 2020/0230054 A1
(43) Pub. Date: Jul. 23, 2020

(54) SELECTIVE RELEASE SYSTEM FOR TUMOR THERAPEUTIC AGENTS AND TUMOR DIAGNOSTIC AGENTS AND BIOSENSOR FOR TUMOR TISSUE

(71) Applicants: Fraunhofer-Gesellschaft zur Forderung der angewandten Forschung e.V., Muenchen (DE); Universitaet des Saarlandes, Saarbruecken (DE)

(72) Inventors: Heiko ZIMMERMANN, Waldbrunn (DE); Hagen VON BRIESEN, Huenstetten (DE); Andre SCHULZ, Saarbruecken (DE); Linda ELBERSKIRCH, Saarbruecken (DE); Sylvia WAGNER, Neunkirchen (DE); Benjamin FISCHER, Sulzbach (DE)

(21) Appl. No.: 16/482,697

(22) PCT Filed: Jan. 18, 2018
(86) PCT No.: PCT/EP2018/051176

§ 371 Date: Jul. 31, 2019

(30) Foreign Application Priority Data
Feb. 1, 2017 (DE) 10 2017 000 896.5

(51) Int. Cl. A61K 9/06; A61K 47/02; A61K 47/36; A61K 9/14; A61K 45/00; C12Q 1/26; A61K 9/48; A61K 9/00

(52) U.S. Cl. CPC A61K 9/06; A61K 47/02; A61K 47/36; A61K 9/146; A61K 9/0053; C12Q 1/26; A61K 9/4816; A61K 9/0031; A61K 45/00

(57) ABSTRACT

The present invention relates generally to a sensor for tumor tissue and a local release system for releasing therapeutic agents/diagnostic agents for targeted tumor therapy and diagnosis. More specifically, the invention relates to a cation-stabilised biopolymer gel, in particular an alginate gel, for use as a carrier matrix for a tumor therapeutic agent and/or tumor diagnostic agent, characterised in that the gel destabilises in the presence of a stimulus, in particular lysyl oxidase (LOX), generated by tumor cells or tumor tissue and the tumor therapeutic agent and/or tumor diagnostic agent can be released, and a pharmaceutical composition that comprises this cation-stabilised biopolymer gel with incorporated tumor therapeutic agent and/or tumor diagnostic agent and optionally an additional reporter substance. An associated aspect of the invention relates to an in vitro method for determining the presence and/or amount of a tumor-specific product in the body of a patient, characterised in that a physiological sample from a patient to whom a pharmaceutical composition according to the invention has been administered is examined to ascertain the presence and/or amount of a reporter substance that has been released after destabilisation of the gel as a result of contact with the tumor-specific product to be determined, and optionally compared with reference values.

### BACKGROUND

The invention relates inter alia to a sensor for tumor tissue and a local release system of therapeutic agents for targeted tumor therapy.

Methods of tumor detection which are already known are both image-producing methods such as computed tomography, positron emission tomography, scintigraphy, sonography and endoscopy as well as laboratory tests of tumor markers in bodily fluids such as blood and urine. However, it can only be determined with certainty on the basis of cell and tissue samples whether a suspicious object contains cancer cells. This is associated with an onerous operative removal of samples (biopsy, puncture).

Pharmaceutical compositions and agents for the targeted release of tumor therapeutic agents in tumor tissues are in principle known. However, there is still the problem of reliable identification of tumor tissue and differentiation from healthy tissue and the inherent side effects and bodily impairments to the patient.

The object underlying the present invention is therefore to provide new means with which tumor tissues can be locally and specifically identified or means for specific/selective release of tumor therapeutic agents in a specific tumor target tissue.

These objects are achieved by providing the cation-stabilized biopolymer gel according to claim 1 or the pharmaceutical composition according to claim 13 and the detection method according to claim 17.

Further aspects and preferred embodiments of the invention are the subject matter of the further claims.

### DESCRIPTION OF THE INVENTION

The present invention initially makes use of the fact that various substances, including tumor therapeutic agents, tumor diagnostic agents, reporter substances, etc., can be incorporated in cation-stabilized carrier matrices which are destabilized in the presence of a specific external stimulus and then release the incorporated substances, and is furthermore based on the realization that suitable stimuli are generated and secreted in tumor tissue.

One aspect of the present invention correspondingly relates to a cation-stabilized biopolymer gel for use as a carrier matrix for a tumor therapeutic agent and/or tumor diagnostic agent which is characterized in that the gel can be destabilized in the presence of a stimulus generated by tumor cells or tumor tissue and the tumor therapeutic agent and/or tumor diagnostic agent can be released.

Cation-stabilized biopolymer gels which are suitable for the present invention are typically formed by cross-linking/gelling the gel-forming components in the presence of a sufficiently high concentration of cations, typically divalent cations such as Cu²⁺ ions, Zn²⁺ ions, Ca²⁻ ions or a combination of these, and the gel is destabilized, for example liquefied, upon removal of part or all of the cations from the gel.

Investigations of the inventors led to the surprising realization that the enzyme lysyl oxidase (LOX), which is secreted by very many or even all types of tumors, has such a high affinity to Cu²⁺ ions that a copper ion-stabilized biopolymer gel, e.g. an alginate gel, is destabilized in the presence of LOX by copper ion removal and releases any incorporated substances.

As a result of an elevated expression of LOX by tumor tissue, on one hand, tumor therapeutic agents incorporated in the gel can be released specifically at or in the tumor tissue, and on the other hand the destabilization of the gel and where applicable the release of reporter substances shows the presence of LOX and thus the presence of tumor tissue.

In the case of the cation-stabilized gel used according to the invention, a co-stabilization by further components is also possible. This co-stabilization could be effected e.g. by a) an ionic cross-linking with more than one cation class, e.g. Cu²⁺ and Ca²⁺, and b) by a covalent cross-linking by the polymerization of monomers with more than one double bond and/or by the polycondensation of monomers with more than one functional group, e.g. aldehydes.

The cation-stabilized gel according to the invention can accordingly also optionally have yet further structure-forming components, in particular monomer and/or cross-linking agent components.

More specifically, the further components can be selected from the group of stabilizing cations, monomers with more than one double bond and monomers with more than one functional group.

In a yet more specific embodiment of the cation-stabilized gel, at least one component, in particular a monomer or polymer component, is chemically modified, e.g. provided with coupled-on side groups. The side groups can be selected, for example, from the group of amides, esters and sulphates.

The cation-stabilized biopolymer gel is preferably an alginate gel or an alginate matrix, respectively.

The characteristics of the biopolymer alginate and the resulting gelling properties are directly adjustable by means of various parameters. As a result of the controllable structural properties of the alginate monomers and as a result of concentration variations of polymer, cross-linking agent and/or fluid, typical gel attributes such as, for example, mechanical stability can be influenced. Mixtures and/or chemical modifications, including copolymerizations, substance or cell inclusions and covalent binding reactions to the alginate and/or its matrix surface are furthermore possible without any problems.

In a yet more specific embodiment, the cation-stabilized alginate gel is characterized in that the alginate gel contains Cu²⁺ ions in a concentration of 1 mM to 500 mM, preferably of 1 mM to 100 mM.

The alginate solution used for the production of the gel typically has a viscosity in the range of 1-100 or 1-50 mPas. The viscosity could, however, also be higher.

The viscosity is preferably higher than approximately 15 mPas, it can be, for example, in a range from 16-50 mPas, without, however, being restricted hereto.

In one embodiment, a solution of highly viscous alginates (e.g. approx. 0.65% w/v) is preferably used. In this case, the viscosity is preferably greater than 15 mPas. Alternatively, however, low-viscosity alginates with a viscosity of approximately 1-5 mPas (at a concentration of preferably 2-3% w/v) can also be used.

The cation-stabilized gel can in principle be present in any form which is suitable for the incorporation of desired substances which are to be released later, e.g. therapeutic agents, diagnostic agents, reporter substances. The gel is preferably present in the form of a capsule or a coating.

As already stated above, the stimulus is preferably lysyl oxidase (LOX), it can, however, also be another metal-dependent secretion product of a tumor, for example, a metalloprotease, in particular a metalloprotease which comprises Zn ions.

The tumor to be detected or treated is in principle not particularly restricted. More specifically, it can be selected from the group which comprises tumors of the digestive organs, in particular stomach cancer, small intestine cancer, colon cancer, rectal cancer and anal cancer.

The tumor therapeutic agent is also not particularly restricted. More specifically, the tumor therapeutic agent is selected from the group which comprises dendritic cells, alkylating agents, antimetabolites, podophyllotoxin derivatives, topoisomerase I/II inhibitors, vinca alkaloids, immunomodulatory agents, low-molecular weight kinase inhibitors (sm-KIs), mTOR inhibitors.

In the event that the molecular size of a desired tumor therapeutic agent/diagnostic agent is smaller than the pore size of the gel matrix, the tumor therapeutic agent/diagnostic agent can be bound to larger units, e.g. particles, polymers, by means of covalent or non-covalent interactions.

A further aspect of the present invention relates to a pharmaceutical composition which comprises a cation-stabilized biopolymer gel as defined above as a carrier matrix as well as at least one tumor therapeutic agent and/or at least one tumor diagnostic agent.

This pharmaceutical composition can furthermore still comprise at least one detectable reporter substance which is released upon destabilization of the gel.

In more specific embodiments of this pharmaceutical composition, the reporter substance is selected from the group which comprises dyes or fluorescent markers, e.g. cyanine dyes, food dyes which dye urine, e.g. betanin, B vitamins, methylene blue; as well as particles which are recoverable in the stool.

In the event that the molecule size of a desired reporter substance is smaller than the pore size of the gel matrix, the reporter substance can be bound to larger units, e.g. particles, polymers, by covalent or non-covalent interactions.

The pharmaceutical composition is preferably formulated for oral or rectal administration.

One particular advantage of the release system according to the invention or the pharmaceutical composition according to the invention resides in the fact that the localization of the tumor to be treated does not have to be known precisely in order to be effectively treated.

A yet further aspect of the present invention relates to a method, in particular an in vitro method, for detecting the presence and/or quantity of a tumor-specific product, in particular lysyl oxidase, comprising the contacting of a cation-stabilized biopolymer gel as defined above with tumor cells or tumor tissue, wherein the gel is fully or partially destabilized in the presence of the tumor-specific product and in dependency of the quantity of this tumor-specific product, and the extent of destabilization is detected and optionally compared with reference values.

In a more specific embodiment of this method, the biopolymer gel contains at least one reporter substance, e.g. a dye or a fluorescent marker which is released upon destabilization of the gel and/or undergoes a detectable change in properties, e.g. a change in color, a change in the fluorescence emission or absorption wavelength, a change in fluorescence life span which indicates the extent of destabilization of the gel.

In this method, the detection of the complete or partial destabilization of the gel can be effected by direct visual observation or by a spectroscopic or spectrometric method, in particular selected from the group of VIS spectroscopy, fluorescence spectroscopy, time-resolved fluorescence spectroscopy, FRET spectroscopy, etc.

A further related aspect of the invention relates to an in vitro method for detecting the presence and/or quantity of a tumor-specific product in the body of a patient, which is characterized in that a physiological sample, e.g. blood, urine, stool, of a patient, to whom a pharmaceutical composition for tumor therapy as defined above was administered, is examined for the presence and/or the quantity of a reporter substance, which was released after the destabilization of the gel as a result of contact with the tumor-specific product to be detected, and where applicable compared with reference values.

A correspondingly reduced release would indicate success of the therapy.

### BRIEF DESCRIPTION OF THE FIGURES

FIG. 1 schematically shows the principle of the sensor or selective release system according to the invention.

FIG. 2 shows the release of a reporter substance (FITC dextran) from a copper/alginate matrix after addition of lysyl oxidase.

FIG. 3 shows the reduction in the mechanical stability of a copper/alginate matrix after addition of lysyl oxidase.

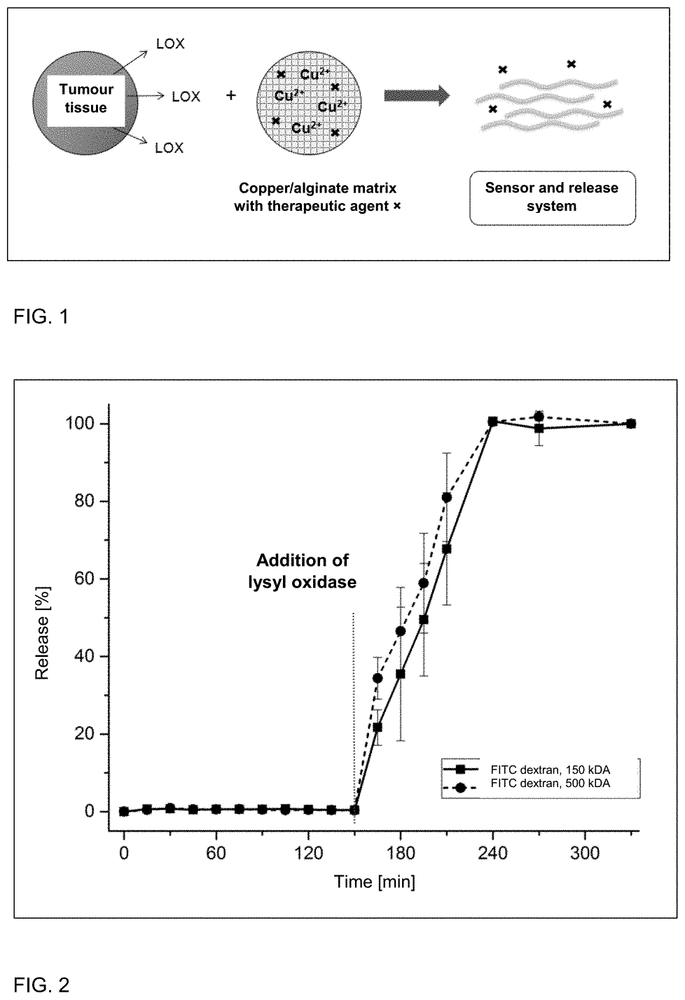

### EXAMPLE 1

### Preparation of an Alginate/Copper Matrix

The alginate/copper matrix was prepared using the following reactants and parameters:
NaCl solution:
   0.9% (w/v) in Aqua dest.
   Osmolarity: 290-300 mOsmol
   pH=7.0-7.5
Alginate solution:
   0.65% (w/v) in 0.9% (w/v) aqueous NaCl solution
   1:1 mixture of alginates from the algae Lessonia trabeculata and Lessonia nigrescens
   Viscosity>15 mPas
CuSO₄.5H₂O solution
   20 mM in Aqua dest.
   Osmolarity: 290-300 mOsmol

The alginate solution was cross-linked with the cross-linking agent Cu²⁺ (in the form of the CuSO₄.5H₂O solution) substantially as described in published US patent application No. 20050158395 A1 (Device and method for producing a cross-linked substance, especially in the form of a microcapsule or layer; Ulrich Zimmermann, Heiko Zimmermann, 2003).

Gel particles or gel capsules with a diameter in a size range of 30-1000 μm are typically generated in this case.

### EXAMPLE 2

### LOX-Induced Destabilization of an Alginate/Copper Matrix and Release of an Incorporated Reporter Substance

The copper/alginate matrix was prepared substantially as described in Example 1. Here, a fluorescent reporter substance, FITC dextran with a molar mass of 150 kDa or 500 kDa, was added to the alginate solution and the reporter substance was incorporated directly into the forming gel.

The destabilization of the alginate/copper matrix and the corresponding release of the reporter substance were effected by adding LOX at a temperature of 37° C. and atmospheric pressure.

The complete or partial destabilization of the gel and the release of the fluorescent reporter substance were detected by VIS spectroscopy.

FIG. 2 shows the time progression of an experiment to release the reporter substance FITC-Dextran. LOX was added after a time span of 150 min. to the suspension in a concentration of 0.31 μM. The release was concluded 90 min after the addition of LOX.

### EXAMPLE 3

### LOX-Induced Destabilization of an Alginate/Copper Matrix and its Detection by Determining the Modulus of Elasticity of the Matrix

The copper/alginate matrix was prepared in terms of reactants and parameters as described in Example 1 as a surface-fixed gel layer.

The destabilization of the alginate/copper matrix was effected by adding LOX at a temperature of 37° C. and atmospheric pressure.

The complete or partial destabilization of the gel was detected by determining the modulus of elasticity of the matrix.

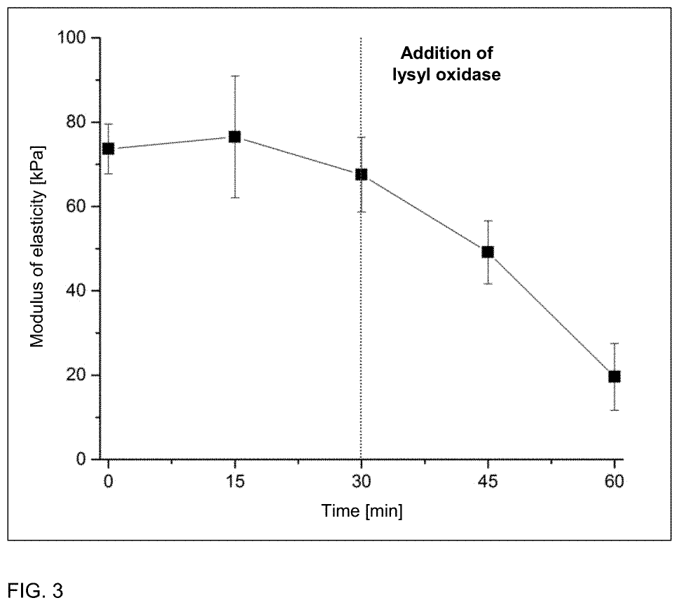

FIG. 3 shows the reduction in the mechanical stability of the copper/alginate matrix after the addition of lysyl oxidase. After 30 min incubation with 0.31 μM LOX, the gel stability was reduced by 75%.

1. A cation-stabilized biopolymer gel for use as a carrier matrix for a tumor therapeutic agent and/or tumor diagnostic agent, wherein the gel is capable of being destabilized in a presence of a stimulus generated by tumor cells or tumor tissue such that the tumor therapeutic agent and/or tumor diagnostic agent is released.

2. The cation-stabilized biopolymer gel according to claim 1, wherein the stimulus is lysyl oxidase (LOX), or a metalloprotease.

3. The cation-stabilized biopolymer gel according to claim 1, wherein tumor cells are, or the tumor tissue is, from tumors of the digestive organs.

4. The cation-stabilized biopolymer gel according claim 1, wherein the tumor therapeutic agent is a member selected from the group consisting of dendritic cells, alkylating agents, antimetabolites, podophyllotoxin derivatives, topoisomerase I/II inhibitors, vinca alkaloids, immunomodulatory agents, low-molecular weight kinase inhibitors (sm-KIs), and mTOR inhibitors.

5. The cation-stabilized biopolymer gel according to claim 1, wherein cations which stabilize the gel are selected from the group consisting of Cu²⁺ cations, Zn²⁺ cations, Ca²⁺ cations and a combination of these cations.

6. The cation-stabilized biopolymer gel according to claim 1, wherein the gel is an alginate gel.

7. The cation-stabilized gel according to claim 6, wherein the alginate gel contains Cu²⁺ ions in a concentration of 1 mM to 500 mM.

8. The cation-stabilized gel according to claim 1, wherein the gel comprises further structure-forming components.

9. The cation-stabilized gel according to claim 8, wherein the further structure-forming components are selected from the group consisting of stabilizing cations, monomers with more than a double bond and monomers with more than one functional group.

10. The cation-stabilized gel according to claim 1, wherein at least one component is provided with coupled side groups.

11. The cation-stabilized gel according to claim 10, wherein the side groups are selected from the group consisting of amides, esters and sulphates.

12. The cation-stabilized gel according to claim 1, wherein the gel is present in a form of a capsule or a coating.

13. A pharmaceutical composition, comprising a cation-stabilized biopolymer gel as defined in claim 1 as a carrier matrix as well as at least one tumor therapeutic agent and/or at least one tumor diagnostic agent.

14. The pharmaceutical composition according to claim 13, further comprising at least one detectable reporter substance which is released upon destabilization of the gel.

15. The pharmaceutical composition according to claim 14, wherein the detectable reporter substance is selected from the group consisting of dyes, fluorescent markers, food dyes which dye urine, and particles which are recoverable in stool.

16. The pharmaceutical composition according to claim 13, which is formulated for oral or rectal administration.

17. An in vitro method for detecting the presence and/or a quantity of a tumor-specific product, comprising contacting of a cation-stabilized biopolymer gel as defined in claim 1 with tumor cells or tumor tissue, wherein the gel is fully or partially destabilized in the presence of the tumor-specific product and in dependency of the quantity of the tumor-specific product, and an extent of destabilization is detected and optionally compared with reference values.

18. The method according to claim 17, wherein the biopolymer gel contains at least one reporter substance, which is released upon destabilization of the gel and/or undergoes a detectable change in properties, which indicates the extent of destabilization of the gel.

19. The method according to claim 17, wherein the detection of the complete or partial destabilization of the gel is effected by direct visual observation or by a spectroscopic or spectrometric method.

20. An in vitro method for detecting a presence and/or a quantity of a tumor-specific product in a body of a patient, wherein a physiological sample to which a pharmaceutical composition for tumor therapy according to claim 14 was administered, is examined for the presence and/or the quantity of a reporter substance, which was released after a destabilization of the gel as a result of contact with the tumor-specific product to be detected, and optionally compared with reference values.

21. The cation-stabilized biopolymer gel according to claim 2, wherein the metalloprotease comprises Zn ions.

22. The cation-stabilized biopolymer gel according to claim 3, wherein the tumor cells are, or the tumor tissue is, from a member selected from the group consisting of stomach cancer, small intestine cancer, colon cancer, rectal cancer and anal cancer.

23. The cation-stabilized biopolymer gel according to claim 18, wherein the detectable change of properties is a member selected from the group consisting of a change in color, a change in a fluorescence emission, a change in an absorption wavelength, and a change in a fluorescence life span.

24. The cation-stabilized biopolymer gel according to claim 19, wherein the spectroscopic or spectrometric method is selected from the group consisting of VIS spectroscopy, fluorescence spectroscopy, time-resolved fluorescence spectroscopy, and FRET spectroscopy.
